(19) United States
(12) Patent Application Publication
(10) Pub. No.: US 2026/0273003 A1
(43) Pub. Date: Sep. 17, 2026

(54) COMBINATION OF RECOMBINANT ONCOLYTIC VIRUS AND CHECKPOINT INHIBITOR FOR THE TREATMENT OF CANCER

(71) Applicants: VIROGIN BIOTECH (SHANGHAI) LTD., Shanghai (CN); VIROGIN BIOTECH CANADA LTD., Richmond (CA)

(72) Inventors: Jun Ding, Vancouver (CA); Ronghua Zhao, Vancouver (CA); Shah Rahimian, Vancouver (CA); Qian Tan, Vancouver (CA)

(21) Appl. No.: 19/473,465

(22) PCT Filed: Apr. 7, 2023
(86) PCT No.: PCT/CN2023/086901

§ 371 Date: Oct. 7, 2025

(51) Int. Cl. A61K 35/763; A61K 9/00; A61K 39/00; A61P 35/00; C07K 16/28

(52) U.S. Cl. CPC A61K 35/763; A61K 9/0019; A61P 35/00; C07K 16/2818; A61K 2039/505; A61K 2039/54; A61K 2039/545; C12N 2710/16032

(57) ABSTRACT

A pharmaceutical combination where a checkpoint inhibitor is combined with a recombinant oncolytic virus and the use of said combination for the treatment of cancer.

### FIELD OF THE INVENTION

The present invention relates generally to a pharmaceutical combination where a checkpoint inhibitor is combined with an oncolytic virus, and methods for treating and/or preventing cancer using said combination.

### BACKGROUND

Malignant tumors are a serious threat to human life and health. Although a variety of standard treatment options exist, such as surgery, radiotherapy, chemotherapy, targe ted therapy, etc., most patients with advanced tumors still have poor prognosis. At present, tumor immunotherapy has made breakthrough progress in the treatment of tumors. Immune-targeted drug therapy (e.g., immune checkpoint suppression) and immune cell therapy (e.g., chimeric antigen receptor T-cell (CAR-T)) have triggered changes in the field of anti-tumor therapy. However, among the currently approved indications for immune checkpoint inhibitors (“CPIs”), the single-drug effective rate is only about 30% (Jiang et al., 2020, Progress and Challenges in Precise Treatment of Tumors With PD-1/L1 Blockade. Frontiers in Immunology, 11 (March)); while CAR-T therapy mainly only targets Cluster of Differentiation 19 (CD19) and B cell maturation antigen (BCMA) that are highly expressed by B cell tumors. The clinical effectiveness of CAR-T therapy in solid tumors has yet to be confirmed (Long et al., 2018, CAR T Cell Therapy of Non-hematopoietic Malignancies: Detours on the Road to Clinical Success. Frontiers in Immunology, 9 (December), 2740). Thus, there exists a strong need for improvements in immunotherapy to benefit patients suffering from many different types of malignant tumors that respond poorly to the current standard of care.

There is no clinically effective treatment for malignant tumors relapsed after and refractory to standard treatment, and patients with this condition are likely to die so oner due to the extensive tumor metastasis or invasion of important organs. Therefore, these patients have an extremely high unmet need for effective treatment, leading to an urgent need to develop new treatment methods to control the progression of the disease and prolong the survival of patients.

Oncolytic viruses (OV) are wild type or genetically modified viruses which preferentially replicate in cancer cells. OVs can induce anti-tumor efficacy in various ways. Many cellular mechanisms in tumor cells favor viral lytic replication, including defective antiviral responses, upregulated oncogene expression, enhanced DNA repair activity, antiapoptotic responses and increased activity of survival and proliferation pathways. Moreover, OV infection can promote remodeling of the tumor microenvironment into a more immunogenic state (e.g., by recruiting tumor-infiltrating lymphocytes). Genetic engineering of OV can also be used to express exogenous immune-modulating payloads that further promote antitumor immunity.

Many OVs in development possess limited clinical efficacy with no overall survival benefit when applied alone. The poor systemic antitumor response and effect on visceral metastases may be due to two factors: Lack of (or ineffective) immunostimulatory payloads which fail to achieve a systemic antitumor immune effect; and attenuation of the virus which limits viral replication and thus negatively impacts the ability of the virus to spread within a tumor mass.

The antiviral immune response triggered by OVs has often been considered unfavorable for OV-based therapies. However, the initial antiviral response can form the foundation for a clinically meaningful antitumor effect if combined with intratumoral co-expression of properly designed immunomodulatory payloads carried by the OV. (Gujar et. al., 2017). In addition, in the absence of a CPI, tumor cells may be able to escape from the activated immune cells despite the initial impact of the OV. Thus, combinations of OVs with CPIs may have synergistic antitumor effects.

The present invention overcomes shortcomings of current cancer therapies, including immunotherapies, increases viral potency without compromising patient safety, and further provides additional unexpected benefits.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

The present invention provides a pharmaceutical combination containing recombinant oncolytic virus VG2025 and a checkpoint inhibitor. Within certain embodiments of the invention, the checkpoint inhibitor is an anti-PD1 antibody or an anti-PD-L1 antibody. Within certain embodiments of the invention, the checkpoint inhibitor is nivolumab.

Another aspect of the present invention provides a method of treating cancer comprising administering the pharmaceutical combination of the present invention.

A further aspect of the present invention provides a kit which comprises recombinant oncolytic virus VG2025, and an anti-PD1 antibody or an anti-PD-L1 antibody.

The details of one or more embodiments are set forth in the descript ion below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Features of the present disclosure, its nature and various advantages will be apparent from the accompanying drawings and the following detailed descript ion of various embodiments.

FIG. 1 shows tumor size after treatment with mVG2025 combined with anti-PD-1 antibody in mouse colorectal cancer model.

FIG. 2a shows tumor size after treatment with mVG2025 combined with anti-PD-1 antibody in mouse sarcoma model.

FIG. 2b shows the survival curve of mice after treatment with mVG2025 combined with anti-PD-1 antibody.

FIG. 3a shows cellular composition of the left side tumors after treatment with vehicle, mVG2025, anti-PD-1 antibody, and VG2025 combined with anti-PD-1 antibody, respectively.

FIG. 3b shows cellular composition of the right side tumors after treatment with vehicle, mVG2025, anti-PD-1 antibody, and VG2025 combined with anti-PD-1 antibody, respectively.

FIG. 4 shows the result of activation of genes related to tumor-inflammation score in samples from vehicle, mVG2025, anti-PD-1 antibody, and VG2025 combined with anti-PD-1 antibody treatment groups, respectively.

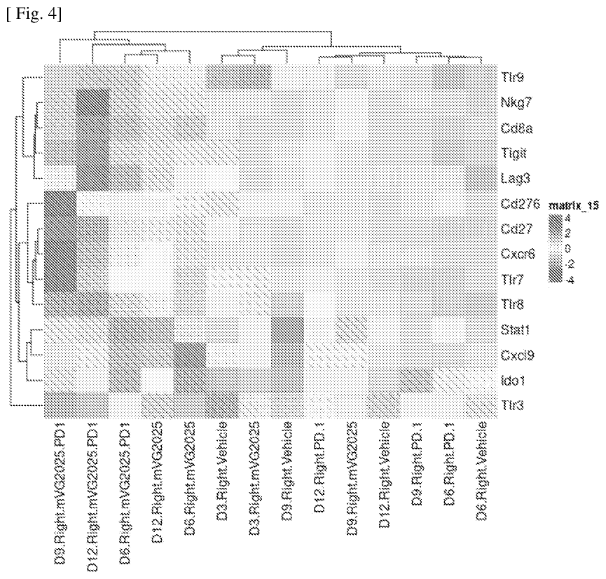

Corresponding reference numerals indicate corresponding parts throughout the drawings.

### DETAILED DESCRIPTION OF THE INVENTION

### Definitions

The term “oncolytic herpes virus” or “oHSV” refers generally to a herpes virus capable of replicating in and killing tumor cells. As used herein, VG2025 refers to a recombinant oHSV as described in international patent ap plication No. WO2022204434A1, filed Mar. 24, 2022, the content of which is incorporated herein by reference in its entirety. “mVG2025” refers to the mou se version of VG2025.

As used herein the term “agent” is understood to mean a substance that produces a desired effect in a tissue, system, animal, mammal, human, or other subject. It is also to be understood that an “agent” may be a single compound or a combi nation or composition of two or more compounds.

“Checkpoint inhibitor” or “CPI” means any agent that cause a blockade of immune system inhibitory checkpoints. Blockade of inhibitory immune checkpoints activates immune system function. In a preferred embodiment the ligand-receptor interaction as a target for cancer treatment is the interaction between the transmembrane programmed cell death 1 protein (PDCD1, PD-1, also known as CD279) and its ligand, PD-1 ligand (PD-L1, CD274). In normal physiology PD-L1 on the cell surface binds to PD-1 on an immune cell surface, which inhibits immune cell activity.

Upregulation of PD-L1 on the cancer cell surface may allow them to evade the patient's immune system by inhibiting T cells that might otherwise attack the tumor cell. Antibodies that bind to either PD-1 or PD-L1 and therefore block the interaction allow the T-cells to attack the tumor. Thus, in a preferred embodiment, PD-1 antagonists are used as CPI.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SL EB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of hum an PD-L1 to human PD-1, and preferably blocks binding of both human PD-L1 and PD-L2 to human PD-1. Human PD-1 amino acid sequences can be found in NCBI Locus N.: NP_054862 and NP_079515, respectively.

PD-1 antagonists useful in any of the treatment methods, medicaments and uses of the present invention include monoclonal antibodies (mAb), or antigen binding fragments thereof, which specifically bind to PD-1 or PD-L1. In some embodiments, the PD-1 antagonist binds to PD-1 or PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions. In some embodiments, the antigen binding fragment is selected from the group consisting of Fab, Fab′-SH, F(ab′) 2, scFv and Fv fragments. Representative examples of PD-1 antagonists include nucleic acid sequences that encode Nivolumab (OPDIVO—a human anti-PD-1 antibody).

In some embodiments, at least one of the therapeutic agents in the pharmaceutical combination of the present invention is administered using the same dosage regimen (dose, frequency and duration of treatment) that is typically employed when the agent is used as monotherapy for treating the same cancer. In other embodiments, the patient receives a lower total amount of at least one of the therapeutic agents in the pharmaceutical combination than when the agent is used as monotherapy, e.g., smaller doses, less frequent doses, and/or shorter treatment duration. The CPI and oncolytic virus described herein may be provided as a kit which may comprise a first container and a second container and a package insert. The first container contains at least one dose of a medicament which may comprise a CPI, preferably an anti-PD-1 antagonist, the second container contains at least one dose of a medicament which may comprise an oncolytic virus, and the package insert, or label, which may comprise instructions for treating a patient for cancer using the medicaments. The first and second containers may be comprised of the same or different shape (e.g., vials, syringes and ampoules) and/or material (e.g. plastic or glass). The kit may further comprise other materials that may be useful in administering the medicaments, such as diluents, filters, IV bags and lines, needles and syringes. In some preferred embodiments of the kit, the anti-PD-1 antagonist is an anti-PD-1 antibody and the instructions state that the medicaments are intended for use in treating a patient having a cancer that tests positive for PD-L1 expression by an IHC assay.

In the present invention it has been shown for the first time that the combinatorial use of VG2025, and a CPI, particularly Nivolumab, may be a valid approach against cancer, wherein the cancer may comprise adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, Ewing's family of tumors (PNET), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic leukemia, oral cavity cancer, liver cancer, lung cancer, non-small cell lung cancer, small cell lymphoma, AIDS-related lymphoma, central nervous system (primary) lymphoma, cutaneous T-cell lymphoma, Hodgkin's disease, non-Hodgkin's disease, malignant mesothelioma, melanoma, Merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, thyroid cancer, urethral cancer, uterine cancer, sarcoma, unusual cancer of childhood, vaginal cancer, vulvar cancer, or Wilms'tumor. Preferred types of cancer to be treated include, but are not limited to, urothelial tumors, non-small-cell lung cancer (NSCLC), cervical cancer, endometrial cancer, gastric cancer, MSI-H and TMB-H tumors, colorectal cancer, pancreatic cancer, sarcoma, and non-melanoma skin cancers that were intolerant to, or progressed, during or after standard of care treatments. Preferably, the combinatorial use of VG2025 and a CPI as described in the present invention is leveraged to treat solid tumors. As outlined in more detail in the examples, a considerable tumor size reduction was surprisingly obtained in tumor types which are normally not responsive to CPI treatment alone, particularly in colorectal cancer and in sarcoma.

### Compositions

The present invention also relates to the use of VG2025 and CPI for the preparation of a pharmaceutical combination or composition for the treatment of cancer.

The mode of administration of VG2025 and CPI may be simultaneously or sequentially, wherein, preferably, VG2025 and CPI are sequentially (or separately) administered. This means that VG2025 and CPI may be provided in a single unit dosage form for being taken together or as separate entities (e.g., in separate containers) to be administered simultaneously or with a certain time difference. This time difference may be between 1 minute and 4 weeks, preferably ≤2 weeks if CPI is administered once per month, or ≤2 days if CPI is administered every 2 weeks. Within some embodiments, VG2025 can be first administered to the subject about every 2 weeks, followed by CPI administration about every 2 weeks at about 240 mg dose of CPI, or about every 4 weeks at about 480 mg dose of CPI. Within additional embodiments, either VG2025 or the CPI or both VG2025 and CPI are administered in a series of infusions for up to two years or more, separated by a time period of anywhere between 1 minute and 4 weeks, and preferably 2 weeks or 4 weeks, between injections.

Preferably, the virus and the CPI are administered as separate compounds. Concomitant treatment with the two agents is also possible. In addition, it is possible to administer the virus via a different route of administration than the CPI. In this regard it may be advantageous to administer the virus intratumorally and the CPI systemically, e.g., orally, subcutaneously, intramuscularly, intraperitoneally, or intravenously. In a particular preferred embodiment, the virus is administered intratumorally, and the CPI is administered intraperitoneally. In another preferred embodiment, the virus is administered intratumorally and the CPI is administered intravenously. Alternatively, both the virus and the CPI may be administered intratumorally, whereupon the dose of CPI will be adjusted according to practical need.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and/or CPI and that is not toxic to the subject to whom it is administered (see generally Remington: The Science and Practice of Pharmacy, Lippincott Williams &Wilkins; 21st ed. (May 1, 2005 and in The United States PharmacopE1A: The National Formulary (USP 40-NF 35 and Supplements).

Non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the oncolytic virus and/or CPI, or any other suitable vehicle, delivery or dispensing means or material (s). Such carriers can be formulated by conventional methods and can be administered to the subject at an effective dose. Additional pharmaceutically acceptable excipients include, but are not limited to, water, saline, polyethylene glycol, hyaluronic acid, and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical grade) carriers, diluents and excipients that may be used to deliver the oncolytic virus and/or CPT to a cancer cell will preferably not induce an immune response in the individual (subject) receiving the composition (and will preferably be administered without undue toxicity).

The compositions provided herein can be provided at a variety of concentrations. For example, dosages of oncolytic virus can be provided which range from about 10⁴ to about 10¹⁰ PFU. Within further embodiments, the dosage can range from about 1×10⁶ to about 1×10⁹ PFU per dose, or from about 1×10⁷ to about 1×10⁹ PFU per dose, or from about 1×10⁸ to about 4×10⁸ PFU per dose, with up to 5 mL, up to 4 mL, up to 3 mL, up to 2 mL, up to 1 mL, up to 0.5 mL, up to 0.2 mL, or up to 0.1 mL administered to a subject, with an interval of about 1 week, about 2 weeks, about 3 weeks, or about 4 weeks between injections. Dosages of CPI can be provided which range from about 0.2 mg/kg to about 20 mg/kg. In certain embodiments, dosages of CPI are administered ranging from about 0.5 mg/kg to about 6 mg/kg. In other embodiments, dosages of CPI are administered ranging from about 1 mg/kg to about 3 mg/kg. CPI may be administered to a subject with an interval of about 1 week, about 2 weeks, about 3 weeks, or about 4 weeks between injections. As used herein, PFU refers to “plaque forming unit”.

The compositions may be stored at a temperature conducive to stable shelf-life and includes room temperature (about 20° C.), 4° C., −20° C., −80° C., and in liquid N₂. Because compositions intended for use in vivo generally do not have preservatives, storage will generally be at colder temperatures. Compositions may be stored dry (e.g., lyophilized) or in liquid form.

### Administration

In addition to the pharmaceutical combination or composition described herein, various methods of using such combination or composition to treat or ameliorate cancer are provided, comprising the step of administering an effective dose or amount of VG2025 and/or CPI as described herein to a subject.

The terms “effective dose” and “effective amount” refers to amount of a pharmaceutical agent that is sufficient to effect treatment of a targeted cancer, e.g., amounts that are effective to reduce a targeted tumor size or load, or otherwise hinder the growth rate of targeted tumor cells. More particularly, such terms refer to amounts that are effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, diminishment of the reoccurrence of disease, and remission (whether partial or total), whether detectable or undetectable. The terms “treating”, and “treatment” can also mean prolonging survival as compared to expected survival if not receiving treatment.

The terms “prevent” or “preventing” mean to lessen the incidence, occurrence, or likelihood of occurrence of a disease.

The term “cancer” refers to an abnormal growth of cells or tissue and is understood to include malignant neoplastic growths. Representative forms of cancer include, but are not limited to adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, Ewing's family of tumors (PNET), extracranial germ cell tumor, eye cancer, intraocular melanoma, gallbladder cancer, gastric cancer, germ cell tumor, extragonadal gestational trophoblastic tumor, head and neck cancer, hypopharyngeal cancer, islet cell carcinoma, laryngeal cancer, leukemia, acute lymphoblastic leukemia, oral cavity cancer, liver cancer, lung cancer, non-small cell lung cancer, small cell lymphoma, AIDS-related lymphoma, central nervous system (primary) lymphoma, cutaneous T-cell lymphoma, Hodgkin's disease, non-Hodgkin's disease, malignant mesothelioma, melanoma, Merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, thyroid cancer, urethral cancer, uterine cancer, sarcoma, unusual cancer of childhood, vaginal cancer, vulvar cancer, or Wilms'tumor.

In the context of cancer, the terms “treat” or “treating” refer to amounts of immunogenic protein (and/or nucleic acid sequence encoding such protein) that induces remiss ion, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The pharmaceutical combination described herein may be given by a route that is e.g., oral, topical, parenteral, systemic, intravenous, intraperitoneal, intramuscular, intraocular, intrathecal, intratumoral, subcutaneous, or transdermal. Within certain embodiments the combination may be administered to the patient from a source implanted in the patient. For example, a cannula or a catheter, e.g., of silicone or other biocompatible material, can be connected to a small sub cutaneous reservoir (Rickham reservoir) installed in the patient during tumor removal or by a separate procedure, to permit the VG2025 composition to be injected locally at various times without further surgical intervention. The virus or derived vectors can also be injected into the tumor by stereotactic surgical techniques or by navigation targeting techniques. The site of administration may be intra-tumor or at a site distant from the tumor. Administration of the virus can also be performed by continuous infusion of viral particles or fluids containing viral particles through implant ed catheters at low flow rates using suitable pump systems, e.g., peristaltic infusion pumps or convection enhanced delivery (CED) pumps. The route of ad ministration will often depend on the type of cancer being targeted.

The optimal or appropriate dosage regimen of the pharmaceutical combination is readily determinable within the skill of the art, by the attending physician based on patient data, patient observations, and various clinical factors, including for example a subject's size, body surface area, age, gender, and the particular combination being administered, the time and route of administration, the type of cancer being treated, the general health of the patient, and other drug therapies to which the patient is being subjected.

The dosage regimen may also allow the clinical use of the virus and/or CPI at lower therapeutic doses, thus preserving or even enhancing anticancer efficacy while increasing safety and reducing and/or avoiding side effects. In view of the strong synergistic effect between the virus and CPI it is possible to foresee a reduction of the therapeutic doses, e.g., about one quarter, about one half, or about one third of the previously used single component doses, while preserving the desired therapeutic effect. In view of the reduced dosage, (severe) side effects may be reduced or even avoided entirely.

The pharmaceutical combination of the invention may be used prior to or following surgery to remove a tumor and may be used prior to, during or after radiation therapy.

The pharmaceutical combination may also comprise one or more additional therapeutic agents. The additional therapeutic agent may be, e.g., a chemotherapeutic agent, a biotherapeutic agent (including but not limited to antibodies to VEGF, EGFR, Her2/neu, VEGF receptors, other growth factor receptors, CD20, CD40, CD40L, CTLA-4, OX-40 4-1BB, and ICOS), an immunogenic ag ent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines (such as but not limited to GM-CSF).

Examples of chemotherapeutic agents include alkylating agents such as cyclosphosphamide, busulfan, a camptothecin, chlorozotocin, fotemustine, lomustine, nimustine, ranimustine, antibiotics, bleomycins, caminomycin, dactinomycin, daunorubicin, idarubicin, 5-flourouracil (5-FU), methotrexate, cytarabine, platinum analog such as cisplatin and carboplatin; vinblastine, platinum; etoposide (VP-16); ifosfamide, mitoxantrone; vincristine; vinorelbine; novantrone; teniposide; edatrexate; daunomycin; aminopterin, xeloda; ibandronate; topoisomerase inhibitors; difluoromethylornithine (DMFO); retinoids, tamoxifen, raloxifene, droloxifene, 4-hydroxytamoxifen, trioxifene, keoxifene or aromatase inhibitors.

A therapeutically effective amount is preferably administered. This is an amount that is sufficient to show benefit to the subject. The actual amount administered, and the time-course of administration will depend at least in part on the nature of the cancer, the condition of the subject, site of delivery, and other factors.

### Clinical Study

The pharmaceutical combination of the present invention may be studied in clinical trials. Without intention of being exhaustive or limiting, exemplified criteria for inclusion, exclusion and removal of subjects in clinical study is provided below.

Subjects will be eligible for study participation if all the following criteria are met:

1) Signed written informed consent.

2) Males or females aged ≥18 years.

3) Performance status: Eastern Cooperative Oncology Group (ECOG) 0 or 1.

4) Subjects with advanced malignant solid tumor which is refractory/relapsed after an d/or intolerant of standard therapies or for which no standard therapy exists or available (refer to National Comprehensive Cancer Network [NCCN] guidelines).

5) At least 1 injectable lesion ≥15 mm in longest diameter and/or nodal lesions that are visible or palpable deemed injectable. Subjects with deep lesions (such as hepatic lesions) that can be safely injected under guided imaging, can be considered for intratumoral injection of VG2025.

6) Seropositive for Herpes Simplex Virus (HSV) (based on serology and positive antibody testing).

7) Had an interval of ≥4 weeks (28 days) since exposure to immunotherapy, an interval of ≥3 weeks (21 days) since exposure to systemic chemotherapy, an interval of ≥6 weeks (42 days) since exposure to nitrosourea, and an interval of ≥4 weeks (28 days) since exposure to radiotherapy, prior to dosing.

8) Subjects must also:
a) have either disease progression or intolerable toxicity after standard treatment (due to either disease progression or intolerable toxicity),
b) have received at least one prior line of systemic standard therapy,
c) be fully recovered or at a grade 1 toxicity (based on CTCAE V5.0) due to prior chemotherapy, immunotherapy, or radiotherapy at Screening.

9) Life expectancy of at least 3 months.

10) Eligibility requirements also include:
a) Hemoglobin ≥90 grams (g)/liter (L),
b) ANC ≥1.5×10\hat{ }9/L,
c) Lymphocyte count ≥1.5×10⁹/L; for lymphocyte count between 0.75×10⁹/L to 1.5×10⁹/L the enrollment should be decided by investigator in discussion with the Medical Monitor,
d) Platelet count ≥100×10⁹/L,
e) Serum creatinine ≤1.5× the upper limit of the reference range or Estimated Glomerular Filtration Rate (eGFR)≥45 mL/min/1.73 m², for subjects with creatinine levels above institutional normal,
f) Serum bilirubin ≤1.5× the upper limit of the reference range,
g) Alanine aminotransferase (ALT) and aspartate aminotransferase (AST) ≤3.0× the upper limit of the reference range, or <5.0× the upper limit of the reference range for subjects with liver metastases,
h) international normalization ratio (INR) or prothrombin time (PT) ≤1.5×ULN, unless the subject is receiving anticoagulant therapy, in which case PT and partial thromboplastin time (PTT)/activated PTT (aPTT) must be within therapeutic range of intended use of anticoagulants,
i) PTT or aPTT≤1.5×ULN unless the subject is receiving anticoagulant therapy as long as PT and PTT/aPTT is within therapeutic range of intended use of anticoagulants.

11) Subjects with dermatoses without active infection will be allowed.

12) Subjects whose baseline pulse oximetry is at least 90% on Room air.

13) Male subjects must abstain from heterosexual activities or agree to use a condom during the study and for 6 months following the end of study. Women of childbearing potential must be willing to abstain from heterosexual activities or agree to use highly effective, double-barrier contraception during the study and for 6 months following the end of study, to avoid pregnancy. Double-barrier contraception is defined as a condom AND one other form of the following:
a) Birth control pills (The Pill),
b) Depo or injectable birth control,
c) Intrauterine Device,
d) Birth control patch (e.g., Ortho Evra),
e) NuvaRing®,
f) Documented evidence of surgical sterilization at least 6 months prior to the Screening visit, i.e., tubal ligation or hysterectomy for women or vasectomy for men.

14) Males must not donate sperm during the study and for at least 6 months after end of the study. Male partners of female subjects and female partners of male subjects must also use contraception, if they are of childbearing potential.

15) Females of childbearing potential must have a negative pregnancy test at Screening and on Day 1.

A subject will be ineligible for study participation if any of the following criteria is met:

1) Participation in any previous trial when the last dose of the investigational agent was received within the last 4 weeks prior to dosing. If the half-life of the investigational drug was less than 5 days, subjects may participate in the study sooner with medical monitor's approval.

2) Tumors to be injected are in mucosal regions or close to an airway, major blood vessel or spinal cord that, in the opinion of the Investigator could cause occlusion or compression in the case of tumor swelling or erosion into a major vessel in the case of necrosis.

3) Subjects with any primary Central Nervous System (CNS) malignancy including glioma and current, active, progressing CNS malignant lesions, including carcinomatosis meningitis are excluded. Subjects with treated brain metastases are eligible if there is no evidence of progression for at least 4 weeks after CNS-directed treatment, as ascertained by clinical examination and brain imaging (magnetic resonance imaging [MRI] or computed tomography [CT] scan) during the screening period and off systemic steroids (for at least 2 weeks prior to first do se of VG2025).

4) Major surgery within 14 days prior to Screening commencement. Procedures such as Pleural drainage, tumor needle biopsy, feeding tube placement and central venous catheter are not considered as major surgery.

5) Intercurrent serious infections within 28 days prior to Screening or treated systematically with intravenous antibiotics within 14 days prior to Screening.

6) Life-threatening illness unrelated to cancer.

7) Active Herpes or COVID-19 infection.

8) Treatment with antiviral agents within 14 days prior to Screening commencement.

9) Subjects with congestive heart failure (as defined by New York He art Association Functional Classification III or IV), unstable angina, serious uncontrolled cardiac arrhythmia, a myocardial infarction within 6 months prior to study entry or a history of myocarditis.

10) Known to test positive for human immunodeficiency virus (HIV), hepatitis B or C virus, or syphilis.

For patients with positive test results of HIV that meet all the following eligibility criteria may be enrolled:
Have a T-cell (CD4+) count ≥350 cells/uL
No History of opportunistic infections or other malignancies
Have an HIV viral load less than 400 copies/mL prior to enrollment
In the opinion of the investigator, their antiretroviral therapy (ART) or other HIV treatments will not interfere with the activity of the investigational product or ca use any confusion with the assessment of the investigational drug toxicities.

For patients with positive test results of HBV or HCV:
Patients with current active HBV or HCV infection or those with a history of HBV or HCV infection that are still on treatment are excluded. Patients with a past history of HBV and HCV infection are only allowed to participate, if they have a negative viral load test. (HBV DNA Polymerase Chain Reaction (PCR) and HCV RNA PCR) within 30 days before screening).

11) Subjects with a condition requiring systemic treatment with either corticosteroids (>10 milligrams (mg) daily prednisone equivalent) or other immunosuppressive medications within 14 days prior to dosing. Inhaled or topical steroids, and adrenal replacement steroid doses, are permitted in the absence of active autoimmune disease.

12) Subjects who have been on systemic anticoagulants and cannot safely hold anticoagulation for planned intratumoral injections and study procedures.

13) Subjects with prior radiation therapy to the tumor lesion to be injected are excluded from the study, unless there is evidence of tumor progression in the most recent imaging or by biopsy, following completion of radiotherapy.

14) Subjects with active or documented history of autoimmune disease within 2 years prior to Screening commencement.

Please note that subjects with vitiligo, resolved childhood asthma/atopy, autoimmune endocrinopathy on stable replacement therapy, or psoriasis not requiring systemic treatment within the past 2 years will be allowed.

15) Subjects with history of primary immune deficiency.

16) Subjects with history of organ transplant that requires use of immunosuppressive medications.

17) Use of any vaccines within 4 weeks prior to dosing.

18) Subjects with any prior ≥Grade 3 immune-related adverse event (irAE) while receiving any previous immunotherapy agent, or any unresolved irAE Grade >1 (as per NCI CTCAE Version 5.0).

19) Subjects with any >Grade 1 toxicity (as per NCI CTCAE Version 5.0) related to prior anti-cancer therapy, except those that are deemed not clinically significant by the Investigator in discussion with the Medical Monitor.

20) Subjects with any clinically significant unrelated systemic illness (serious infections or significant cardiac, pulmonary, hepatic or other organ dysfunction, bleeding tendency), that would compromise the subject's ability to tolerate protocol therapy or would likely interfere with the study procedures or results.

21) Subjects with history of prior HSV-1 encephalitis or encephalitis due to other etiology.

22) Subjects with active infection causing fever (temperature >38.1° C.) or subjects with unexplained fever (temperature >38.1° C.) within 7 days prior to the day of investigational product administration.

A subject may be removed from the study for the following:
Subject elects to withdraw from the study treatment or consent.
Development of an AE, an inter-current illness, or procedural complication, which would interfere with the subject's participation as per PI's judgement.
Newly discovered data that raise concern about the safety of the study drug so that continuation would pose increased risks to the subjects as per PIs judgement.
The PI may discontinue a subject if, in the Investigator's medical judgment, continued participation is not in the subject's best interests.
Sponsor elects to terminate the study prematurely.

For any discontinuation, the Investigator will obtain all the required details and document the date of the premature termination and the main reason for discontinuation in the Case Report Forms (CRFs) and/or electronic CRFs (eCRFs). If the reason for withdrawal is an AE, the specific event or the medical condition associated with main laboratory abnormality will be recorded in the CRF and/or eCRFs.

The Investigator will make every effort to follow any AE until it returns to baseline, judged to be not clinically significant or becomes stable.

A subject who has a DLT event in the first or subsequent cycles will not be permitted to continue on study treatment.

In the combination cohorts, subjects that have to discontinue one drug, can remain on treatment with the other study drug based on PI's discretion and with approval of the medical monitor.

Any subject who discontinues prematurely from the study will undergo an Early Termination visit, with assessments performed for the Early Termination (ET) visit.

The following are some exemplary numbered embodiments of the present disclosure.

1) A pharmaceutical combination containing recombinant oncolytic virus VG2025 and a checkpoint inhibitor, wherein the checkpoint inhibitor is an anti-PD-1 antibody or an anti-PD-L1 antibody.

2) The pharmaceutical combination of embodiment 1, wherein the check point inhibitor is nivolumab.

3) A method of treating cancer comprising administering the pharmaceutical combination of embodiment 1.

4) The method of embodiment 3, wherein the VG2025 and the checkpoint inhibitor are sequentially administered.

5) The method of embodiment 3, wherein the VG2025 and the checkpoint inhibitor are simultaneously administered.

6) The method of embodiment 3, wherein the cancer is a urothelial cancer, non-small-cell lung cancer (NSCLC), cervical cancer, endometrial cancer, gastric cancer, MSI-H and TMB-H tumors, a non-melanoma skin cancer, colorectal cancer, pancreatic cancer, or sarcoma.

7) The method of embodiment 4, wherein the VG2025 is administered intratumorally, and the checkpoint inhibitor is administered systemically.

8) The method of embodiment 7, wherein checkpoint inhibitor is administered intraperitoneally.

9) The method of embodiment 4, wherein the VG2025 and the checkpoint inhibitor are both administered intratumorally.

10) The method of embodiment 4, wherein the VG2025 is administered once.

11) The method of embodiment 4, wherein the VG2025 is administered multiple times.

12) The method of embodiment 11, wherein each dose of the VG2025 is separated by more than 12 hours, more than 1 day or more than 2 days.

13) The method of embodiment 11, wherein each dose of the VG2025 is separated by more than 5 days, more than 1 week, more than 2 weeks, more than 3 weeks or more than 4 weeks.

14) The method of embodiment 10 or 11, wherein the checkpoint inhibitor is administered once.

15) The method of embodiment 10 or 11, wherein the checkpoint inhibitor is administered multiple times.

16) The method of embodiment 15, wherein each dose of the checkpoint inhibitor is separated by more than 12 hours, more than 1 day or more than 2 days.

17) The method of embodiment 15, wherein each dose of the checkpoint inhibitor is separated by more than 5 days, more than 1 week, more than 2 weeks, more than 3 weeks or more than 4 weeks.

18) The method of embodiment 3, wherein the VG2025 is provided at 1×10⁸ PFU per dose to 1×10⁹ PFU per dose.

19) The method of embodiment 3, wherein the checkpoint inhibitor is provided at 1 mg/kg to 3 mg/kg.

20) A kit which comprises a first container, a second container and a package insert, wherein the first container comprises at least one dose of a pharmaceutical composition containing recombinant oncolytic virus VG2025, the second container comprises at least one dose of a pharmaceutical composition comprising an anti-PD-1 antibody or an anti-PD-L1 antibody, and the package insert comprises instructions for treating an individual having cancer using the pharmaceutical composition.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y,” and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

“Isolated” nucleic acid molecule means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comp rising” a particular nucleotide sequence does not encompass intact chromosomes. Isola ted nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may b e. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and no vel characteristics of the claimed invention.

Any headings used within this document are only being utilized to expedite its review by the reader and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U. S. patents, U. S. patent application publications, U. S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. Applicants reserve the right to physically incorporate into this specification any and all materials and information from any such patents, publications, scientific articles, web sites, electronically available information, and other referenced materials or documents.

The publications discussed above and throughout the text are provided solely for their disclosure prior to the filing date of the present application. Nothing herein is to be construed as an admission that the inventors are not entitled to antedate any referenced publication by virtue of prior invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not 1 imited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verb a in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The Examples and preparations provided below further illustrate and exemplify the compounds of the present invention and methods of preparing such compounds. It is to be understood that the scope of the present invention is not limited in any way by the scope of the following Examples and preparations. In the following Examples, molecules with a single chiral center, unless otherwise note d, exist as a racemic mixture. Those molecules with two or more chiral centers, unless otherwise noted, exist as a racemic mixture of diastereomers. Single enantiomers/diastereomers may be obtained by methods known to those skilled in the art. The starting materials and various reactants utilized or referenced in the examples may be obtained from commercial sources, or are readily prepared from commercially available organic compounds, using methods well-known to one skilled in the art.

### EXAMPLES

### Example 1

Evaluation of efficacy of mVG2025 and anti-PD-1 combo in the MC-38 mouse colorectal cancer model

### 1. Study Objective

The objective of this study is to evaluate the efficacy of mVG2025 in combination with anti-PD-1 antibody in mice implanted with MC-38 murine colorectal tumor cells.

### 2. Experimental Design

TABLE 1
Experimental Design of Efficacy Study

| Group | Article | Dose | N |
|---|---|---|---|
| G1 | Vehicle | 0 | 8F |
| G2 | PD-1 | 10 mg/kg, biw, IP | 8F |
| G3 | mVG2025 + PD1 | 2.4 × 10\hat{ }5 PFU/mouse, Once, Day 1 (“D1”), IT + PD-1, | 8F |
| | | 10 mg/kg, biw, IP | |
| G4 | mVG2025 | 2.4 × 10\hat{ }5 PFU/mouse, Once, D1, IT | 8F |

Note:
N represents the number of animals per group, F represents female; biw represents twice weekly; dosing volume was 100 μL/mouse.
mVG2025 was injected intratumorally (“IT”) under anesthesia. PD-1 antibody was administered intraperitoneally (“IP”). Animals In G3 were given PD-1 antibody 48 h after the first administration of mVG2025 (i.e., PD-1 antibody starts from D3) .

### 3. Study Results

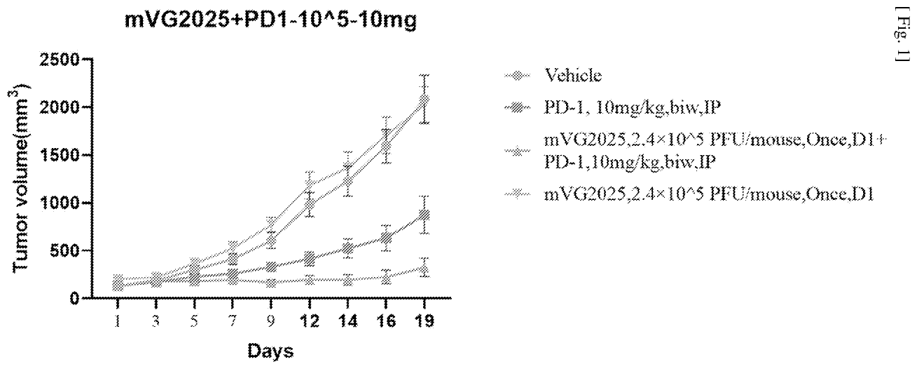

The tumor volumes after treatment are shown in FIG. 1. The group dosed with 2.4×10\hat{ }5 PFU/mouse of mVG2025 combined with anti-PD-1 antibody (10 mg/kg) exhibited significant reduction in tumor growth on day 19 (P<0.01).

### Example 2

Evaluation of efficacy of mVG2025 and anti-PD-1 combo in the WEHI-164 mouse sarcoma model

### 1. Study Objective

The objective of this study is to evaluate the efficacy of mVG2025 in combination with anti-PD-1 antibody in mice implanted with WEHI-164 murine sarcoma tumor cells.

### 2. Experimental Design

TABLE 2
Experimental Design of Efficacy Study

| Group | Article | Dose N | N |
|---|---|---|---|
| G1 | Vehicle | 0 | 8F |
| G2 | PD-1 | 5 mg/kg, biw, IP | 8F |
| G3 | mVG2025 + | 2.4 × 10\hat{ }6 PFU/mouse, triple, Day 0 (“D0”), Day 1 (“D1”), | 8F |
| | PD-1 | Day 2 (“D2”) + PD-1, 5 mg/kg, biw, IP | |
| G4 | mVG2025 | 2.4 × 10\hat{ }6 PFU/mouse, triple, D0, D1, D2 | 8F |

Note:
N represents the number of animals per group, F represents female; biw represents twice weekly; dosing volume was 100 μL/mouse.
mVG2025 was injected intratumorally (“IT”) under anesthesia. PD-1 antibody was administered intraperitoneally (“IP”). Animals In G3 were given PD-1 antibody 24 h after the last administration of mVG2025 (i.e., PD-1 antibody starts from D3) .

### 3. Study Results

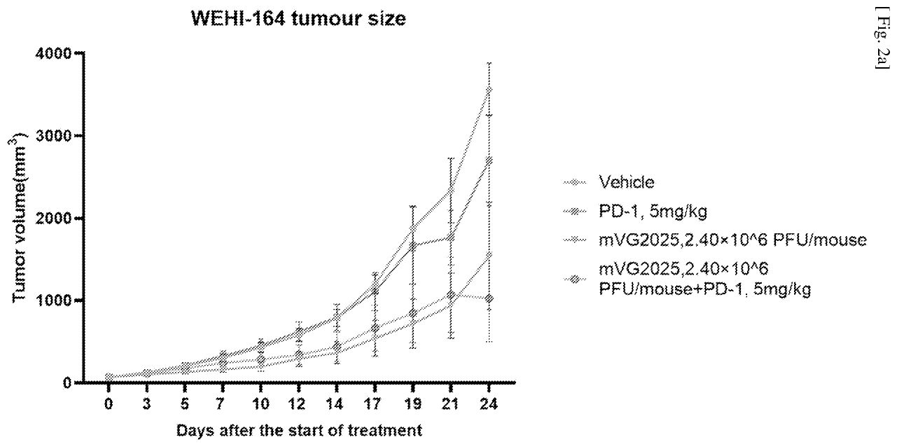

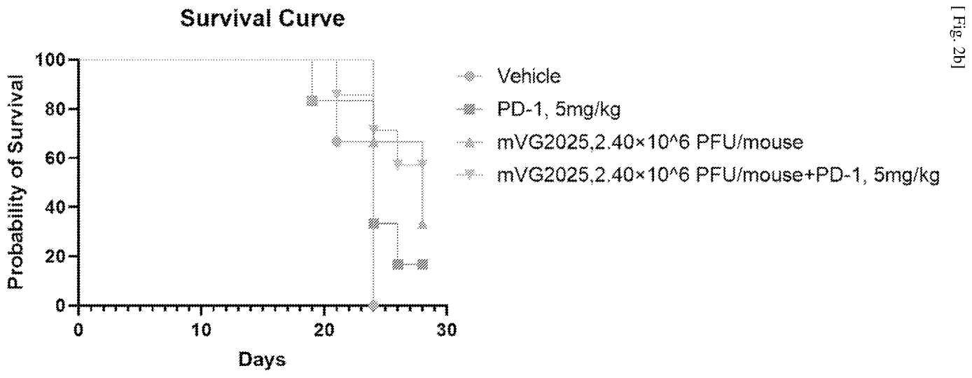

The tumor volumes after treatment are shown in FIG. 2a. The group dosed with 2.4×10\hat{ }6 PFU/mouse of mVG2025 combined with anti-PD-1 antibody (5 mg/kg) exhibited significant reduction in tumor growth on day 24 (P<0.05).

The survival curve results are shown in FIG. 2b. The survival data demonstrates the benefits of the combination treatment. 60% of the mice in the combination treated group were still alive on day 28. In the vehicle treated group, all mice were euthanized due to tumor size reaching a humane endpoint. In the anti-PD-1 antibody treated group and the mVG2025 treated group, less than 40% of the mice were still alive on day 28.

### Example 3

### RNAseq and Mechanistic Studies

Tumor samples from mice treated with either mVG2025, anti-PD-1 antibody, or VG 2025 and anti-mPD-1 antibody combo (combination) were collected for R NAseq analysis. The treatment plan details are described in Table 3. Briefly, MC-38 tumor cells were implanted in both flanks of C57B/6 mice and were allowed to grow up to about 100 mm³ in volume. Mice were then treated with either 2 doses of VG2025 (2.4×10\hat{ }7 PFU) on days 1 and 2, or with vehicle control (phosphate buffer saline, PBS). OV treatment was applied only to the right-side tumor, while the left side was left untreated. Mice were then treated either with 2 doses of 200 μg of anti-PD-1 antibody on days 3 and 7, or with vehicle control (PBS). Tumors were collected from different treatment groups on days 3, 6, 9 and 12.

TABLE 3
Experimental design of RNAseq study.

| | Testing | | | | | | | |
|---|---|---|---|---|---|---|---|---|
| Group | article | Dose | Routine | Frequency | Sample collection | | | |
| G1 | PBS | 0 | IT | D1, D2 | D3 | D6 | D9 | D12 |
| | | | IP | D3, D7 | | | | |
| G2 | PBS | 0 | IT | D1, D2 | N/A | D6 | D9 | D12 |
| | PD-1 | 200 μg/mouse | IP | D3, D7 | | | | |
| G3 | mVG2025 | 2.4 × 10⁷ PFU/mouse | IT | D1, D2 | D3 | D6 | D9 | D12 |
| | PBS | 0 | IP | D3, D7 | | | | |
| G4 | mVG2025 | 2.4 × 10⁷ PFU/mouse | IT | D1, D2 | N/A | D6 | D9 | D12 |
| | PD-1 | 200 μg/mouse | IP | D3, D7 | | | | |

Once the tumors were collected, they were stored in RNAlater solution, and the RNA was isolated from all samples using the Qiagen RNeasy isolation kit. R NAseq analysis was performed using the Illumina NGS platform on different samples to understand the changes exerted by the different treatments in the tumor microenvironment.

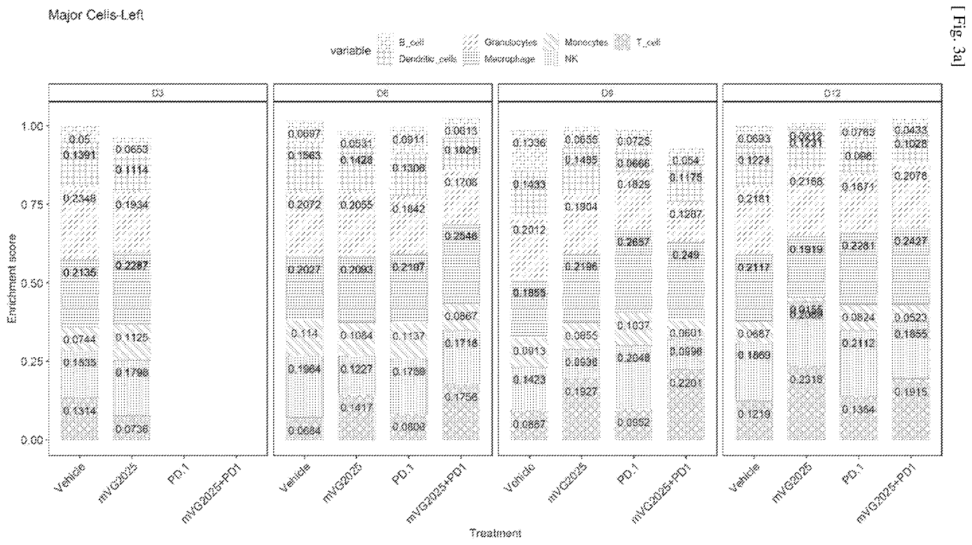

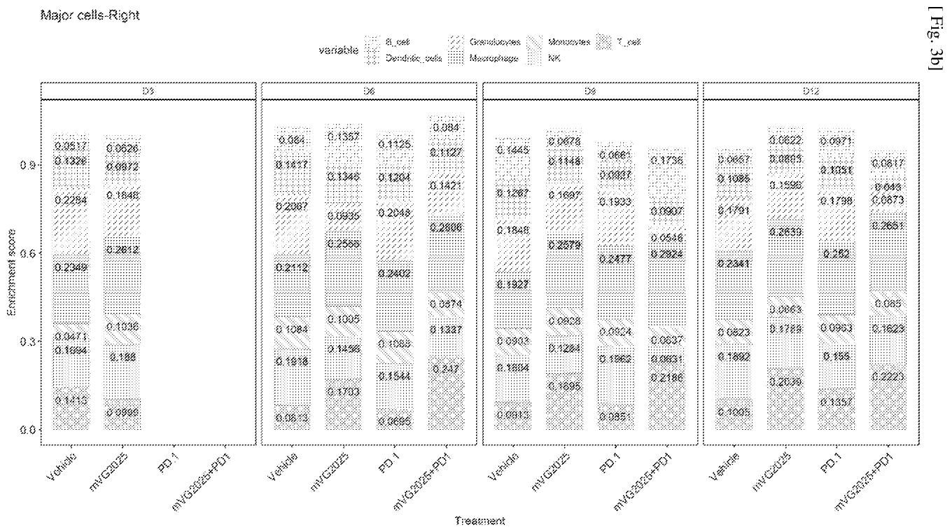

When the cellular composition of the tumors was analyzed (FIG. 3), it was evident that the T-cell population increased in the combination treated group, especially when compared to the group treated with anti-PD-1 antibody alone. This trend was consistent in both the injected tumor (FIG. 3b) and the abscopal/non-injected tumor (FIG. 3a).

When the samples from different treatment groups were clustered based on expression of a panel of genes related to the tumor-inflammation score (Damotte et al. 2019), the combination treated group exhibited the highest activation of this group of genes, indicating that treatment with a combination of mVG2025 and anti-PD-1 antibody effectively activates the genes responsible for the anti-tumor immune response.

All of the U. S. patents, U. S. patent application publications, U. S. patent applications, foreign patents, foreign patent applications and non-patent publications referred to in this specification and/or listed in the Application Data Sheet are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not 1 imited by the disclosure.

1. A pharmaceutical combination containing recombinant oncolytic virus VG2025 and a checkpoint inhibitor, wherein the checkpoint inhibitor is an anti-PD-I antibody or an anti-PD-LI antibody.

2. The pharmaceutical combination of claim 1, wherein the checkpoint inhibitor is nivolumab.

3. A method of treating cancer comprising administering the pharmaceutical combination of claim 1.

4. The method of claim 3, wherein the VG2025 and the checkpoint inhibit or are sequentially administered.

5. The method of claim 3, wherein the VG2025 and the checkpoint inhibit or are simultaneously administered.

6. The method of claim 3, wherein the cancer is a urothelial cancer, no n-small-cell lung cancer (NSCLC), cervical cancer, endometrial cancer, gastric cancer, MSI-H and TMB-H tumors, a non-melanoma skin cancer, colorectal cancer, pancreatic cancer, or sarcoma.

7. The method of claim 4, wherein the VG2025 is administered intratumor ally, and the checkpoint inhibitor is administered systemically.

8. The method of claim 7, wherein checkpoint inhibitor is administered intraperitoneally.

9. The method of claim 4, wherein the VG2025 and the checkpoint inhibit or are both administered intratumorally.

10. The method of claim 4, wherein the VG2025 is administered once.

11. The method of claim 4, wherein the VG2025 is administered multiple times.

12. The method of claim 11, wherein each dose of the VG2025 is separated by more than 12 hours, more than 1 day or more than 2 days.

13. The method of claim 11, wherein each dose of the VG2025 is separated by more than 5 days, more than 1 week, more than 2 weeks, more than 3 weeks or more than 4 weeks.

14. The method of claim 10, wherein the checkpoint inhibitor is ad ministered once.

15. The method of claim 11, wherein the checkpoint inhibitor is ad ministered multiple times.

16. The method of claim 15, wherein each dose of the checkpoint inhibitor is separated by more than 12 hours, more than 1 day or more than 2 days.

17. The method of claim 15, wherein each dose of the checkpoint inhibitor is separated by more than 5 days, more than 1 week, more than 2 weeks, more than 3 weeks or more than 4 weeks.

18. The method of claim 3, wherein the VG2025 is provided at I×I0⁸ PFU per dose to I×I0⁹ PFU per dose.

19. The method of claim 3, wherein the checkpoint inhibitor is provided at 1 mg/kg to 3 mg/kg.

20. A kit which comprises a first container, a second container and a package insert, wherein the first container comprises at least one dose of a pharmaceutical composition containing recombinant oncolytic virus VG2025, the second container comprises at least one dose of a pharmaceutical composition comprising an anti-PD-1 antibody or an anti-PD-LI antibody, and the package insert comprises instructions for treating an individual having cancer using the pharmaceutical composition.
